(19) United States
(12) Patent Application Publication
(10) Pub. No.: US 2019/0259302 A1
(43) Pub. Date: Aug. 22, 2019

(54) TRAINER DEVICE FOR AN AUTOINJECTOR

(71) Applicant: WOCKHARDT LIMITED, Chikalthana (IN)

(72) Inventor: LOGANATHAN SHANMUGAM, Bangalore (IN)

(73) Assignee: WOCKHARDT LIMITED, Chikalthana (IN)

(21) Appl. No.: 16/275,090

(22) Filed: Feb. 13, 2019

(30) Foreign Application Priority Data
Feb. 19, 2018 (IN) IN201821006200
Aug. 21, 2018 (IN) IN201821031340

(51) Int. Cl. G09B 23/28; A61M 5/20

(52) U.S. Cl. CPC G09B 23/285; A61M 2005/2073; A61M 5/2033

(57) ABSTRACT

The invention relates to a trainer device for an autoinjector, comprising: a housing; a collet holder comprising a track; a safety cap removably connected to the collet holder; a cover member; a flex stem having flex arm; a cover member locking mechanism; and a resetting mechanism. The cover member further comprises a plastic sleeve or an elastomeric sleeve to prevent or inhibit the obstruction of the movement of cover member when the user accidentally holds the cover member during the actuation of trainer device.

### PRIORITY DOCUMENTS

This application claims priority from the Indian provisional patent applications IN201821006200 filed on Feb. 19, 2018 and IN201821031340 filed on Aug. 21, 2018, both of which are incorporated herein by reference in their entirety for all purposes.

### FIELD OF INVENTION

The present application is directed to a trainer device for an autoinjector, and particularly, a trainer device wherein the housing is partially received within the cover member.

### BACKGROUND OF THE INVENTION

Autoinjectors have become the main drug delivery devices of choice due to their ability to simplify the number of steps required for injection. However, many patients make mistakes using the devices such as not holding the device correctly or not keeping it in place for long enough. Common errors made by patients include failing to hold the device correctly, not choosing a suitable injection site and not pressing the device hard enough to trigger release of the drug.

To reduce user errors and anxiety, and to build patient confidence, medical device training tools have been developed as trainer devices to support patients in learning how to use their drug delivery devices properly.

Designing and developing an autoinjector trainer can be quite challenging with considerations that do not appear at the surface. The device needs to contain almost all of the elements of the active device, and also include the ability to completely reset the mechanism back to the original state. Not only does the training device need to function within the operational requirements of the active device, but it also needs to perform repeatedly for many cycles depending on the customer requirements.

U.S. Pat. Nos. 5,037,306 and 7,682,155 disclose a training device for an autoinjector.

### SUMMARY

One embodiment discloses a trainer device for an autoinjector comprising: (i) a housing; (ii) a collet holder that includes a track; (iii) a safety cap removably connected to the collet holder; (iv) a cover member; (v) a flex stem having flex arm; and (vi) a cover member locking mechanism having a first locking position and a second locking position, wherein the housing is partially received inside the cover member, and the flex arm of the flex stem traverses the track during actuation of the trainer device upon removal of the safety cap.

Some embodiments disclose a trainer device for an autoinjector comprising: a housing; a cover member; and a cover member locking mechanism comprises cam profiles and cam follower nuts, wherein cam profiles are provided on the cam surfaces of housing and cam follower nuts are positioned between the cover member and the housing in both first locking position and second locking position.

Some embodiments disclose a trainer device for an autoinjector comprising: a housing; a cover member; and a cover member locking mechanism includes cam profiles and cam follower nuts, wherein the cam profile has five vertices and comprises a first angled cam track, a vertical cam track and a second angled cam track, wherein (i) the first angled cam track is formed by traversing of the cam profile from the first vertex to second and third vertices towards the proximal direction; (ii) the vertical cam track is formed by traversing of the cam profile from the third vertex to fourth vertex in the distal direction; and (iii) the second angled cam track is formed by traversing the cam profile from the fourth and fifth vertices to first vertex again in the proximal direction.

Some embodiments disclose a trainer device for an autoinjector comprising: a housing; a cover member; and a cover member locking mechanism includes cam profiles and cam follower nuts, wherein the cam profiles comprise two integral snap lock members which include (i) first integral snap lock member formed by groove cut on the surface of first angled cam track, and (ii) second integral snap lock member formed by groove cut on the surface of vertical cam track.

Some embodiments disclose a trainer device for an autoinjector comprising: a housing; a cover member; and a cover member locking mechanism includes cam profiles and cam follower nuts, wherein the cam follower nuts comprise a cam follower protrusion on the distal end portion; a cam follower button on the proximal end portion; and a cam follower body extending between protrusion and button, wherein the protrusion and button are extending opposite to each other.

Some embodiments disclose a trainer device for an autoinjector comprising: (i) a housing; (ii) a collet holder; (iii) a safety cap removably connected to the collet holder; (iv) a flex stem having flex arm; (v) a cover member, and (vi) a cover member locking mechanism having a first locking position and a second locking position, wherein in the first locking position, the cover member is in the retracted position; and in the second locking position, the cover member is in the extended position; said cover member locking mechanism comprising cam profiles and cam follower nuts, wherein the cam profiles are provided on the cam surfaces of housing and cam follower nuts are positioned between the cover member and the housing in both first locking position and second locking position; and (vii) a resetting mechanism comprising locking projections provided on the housing and reset members provided on the cover member, wherein the resetting of cover member results in proximal movement of cover member to the retracted position; said proximal movement is allowed by pressing the reset members that release the locking connection between the reset members and locking projections.

Some embodiments disclose a trainer device for an autoinjector comprising: a housing; a collet holder that includes a track; a safety cap removably connected to the collet holder; a cover member; and a flex stem having flex arm, wherein the collet holder comprises (i) a single piece body defining longitudinal central axis having opposed upper and bottom surfaces, (ii) opposed open distal end and a closed proximal end, (iii) a pair of laterally opposed sides, and (iv) an upper core member with a top planar surface perpendicular to longitudinal central axis, wherein the upper surface of the collet holder has a stop wall and a recess, wherein the recess extends longitudinally from the open distal end to the stop wall and defining the track for the flex arm.

Some embodiments disclose a trainer device for an autoinjector comprising: a housing and a cover member, wherein the cover member further comprises a sleeve which is at least partially surrounds the outer surface of the cover member to prevent or inhibit the obstruction of the movement of the cover member when the user accidentally holds the cover member during the actuation of trainer device.

Some embodiments disclose a trainer device comprising a plastic sleeve received on the outer surface of the cover member, the plastic sleeve includes a first end and a second end, wherein the first end and the second end are separated by a coil body extending along a coiled axis.

Some embodiments disclose a trainer device comprising an elastomeric sleeve received on the outer surface of the cover member, the elastomeric sleeve includes a first end and a second end, wherein the first end and the second end are separated by a tubular body.

Some embodiments disclose a trainer device for an autoinjector comprising: a housing and a cover member, wherein the cover member comprises a base cover member and a top cover member, and wherein the top cover member has concentrically and coaxially arranged an inner sleeve and an outer sleeve.

### BRIEF DESCRIPTION OF THE DRAWINGS

FIG. 1 shows a side cross-sectional view of the trainer device before actuation;

FIG. 2 shows a side perspective view of the cam profile of the housing;

FIG. 3 shows a side cross-sectional view of the trainer device during removal of the safety cap;

FIG. 4 shows a side cross-sectional view of the trainer device during actuation;

FIG. 5 shows a side cross-sectional view of the trainer device after actuation;

FIG. 6 shows perspective views of the cam follower nut of the trainer device;

FIG. 7 shows perspective views of the flex stem and the flex arm of trainer the device;

FIG. 8 shows a front perspective view of the housing;

FIG. 9 shows perspective views of the collet holder of the trainer device;

FIG. 10 shows perspective views and front views of the top cover member and the bottom cover member of the trainer device according to the embodiment of FIG. 1;

FIG. 11 shows traversing of the cam follower nut in the cam profile of the trainer device during first locking position, actuation stage, second locking position and after resetting stages of operation;

FIG. 12 shows a side cross-sectional view of the trainer device before actuation;

FIG. 13 shows a side cross-sectional view of the trainer device during removal of safety cap;

FIG. 14 shows a side cross-sectional view of the trainer device during actuation;

FIG. 15 shows a side cross-sectional view of the trainer device after actuation;

FIG. 16 shows a perspective view of trainer device wherein the plastic sleeve is partially around the cover member;

FIG. 17 shows perspective views and front views of the top cover member and the bottom cover member of the trainer device according to the embodiment of FIG. 12;

FIG. 18 shows perspective views of the plastic sleeve according to one embodiment;

FIGS. 19a-19e shows assembling procedures of the cover member and the plastic sleeve to the housing of the trainer device according to the embodiment shown in FIGS. 12 to 16;

FIG. 20 shows perspective views of the elastomeric sleeve according another embodiment;

FIG. 21 shows a perspective view of trainer device wherein the elastomeric sleeve is partially around the cover member;

FIG. 22 shows perspective views of the cover member according to yet another embodiment; and

FIG. 23 shows a perspective view of the trainer device according to the cover member of FIG. 22

### DETAILED DESCRIPTION

The present application is directed to a trainer device for an autoinjector,

As used herein, the term “distal end” in the appended figures and description is meant to refer to the end of the trainer device carrying the cover member whereas the term “proximal end” is meant to refer to the opposite end carrying the safety cap. The term “second end” in the appended figures and description is meant to refer to the end of the trainer device carrying the cover member whereas the term “first end” is meant to refer to the opposite end carrying the safety cap.

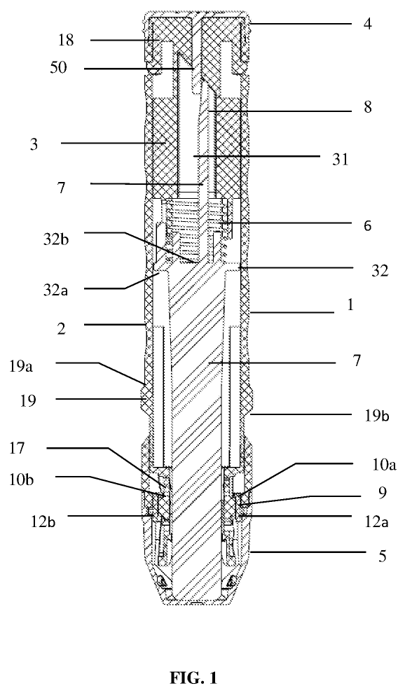

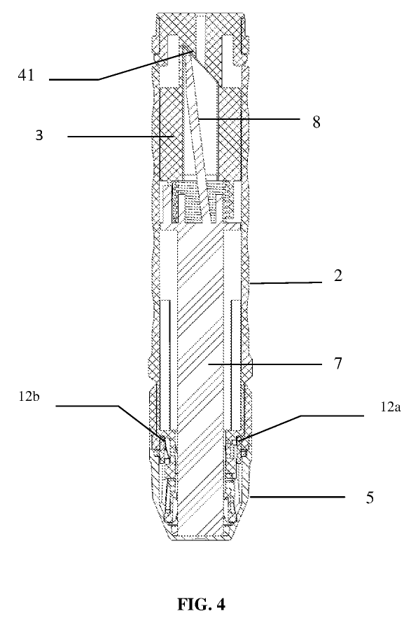

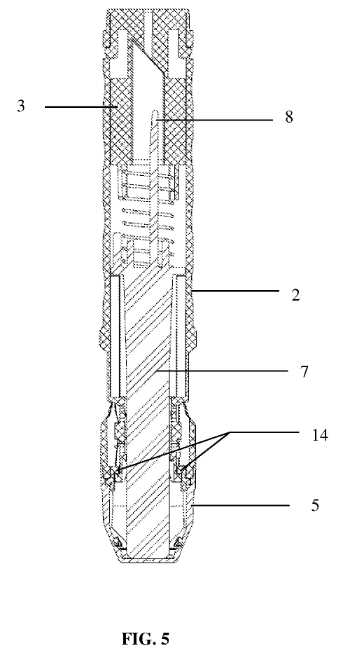

Referring to FIGS. 1-6, a trainer device 1 for an autoinjector, comprising: a housing 2; a collet holder 3, a safety cap 4; a cover member 5; a flex stem 7; a flex arm 8; and a cover member locking mechanism 9, wherein the housing 2 is partially received inside the cover member 5. The cover member locking mechanism 9 comprising diametrically opposed cam profiles 10a, 10b and cam follower nuts 12a, 12b, wherein the cam follower nuts 12a, 12b are positioned between the cover member 5 and the housing 2 in both the first locking position 13 and the second locking position 14. Further the trainer device 1 comprising a spring 6 to bias the flex stem 7; the spring 6 is positioned between flex stem 7 and collet holder 3. The position of cover member 5 in the first locking position 13 and the second locking position 14 may be attained by the cover member locking mechanism 9.

The housing 2 comprising a first sleeve section 16 and a second sleeve section 17, having opposed proximal and distal end portions, the second sleeve section 17 having reduced diameter than the first sleeve section 16 of the housing 2. The first sleeve section 16 at the proximal end has an end surface 18 to receive the safety cap 4 and the distal end having a peripheral ridge 19. The peripheral ridge 19 having front end 19a and rear end 19b portions, whereas the first sleeve section 16 is affixed into the front end portion 19a and the second sleeve section 17 is extending from the rear end portion 19b. At the rear end 19b, a pair of diametrically opposed rectangular shaped like projection heads 20a, 20b, 20c, 20d extending downwardly on the surface of second sleeve section 17.

The second sleeve section 17 at distal end comprising diametrically opposed trapezium shaped like arms 21a, 21b has cutting flank surfaces on the outer surface defining the cam surfaces 22a, 22b. The housing 2 may be cylindrical, oval or elliptical in shape.

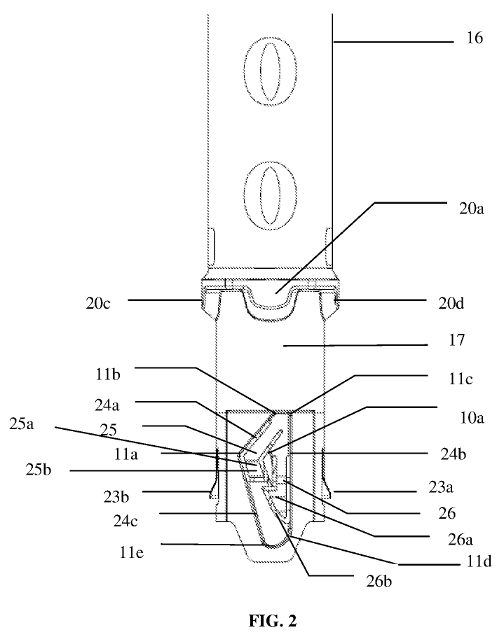

Referring to FIG. 2, the cam profiles 10a, 10b are provided on the diametrically opposed cam surfaces 22a, 22b and each of the cam profile 10a and 10b is shaped like a pentagon having five vertices 11a, 11b, 11c, 11d, and 11e. The cam profiles 10a, 10b comprising a first angled cam track 24a, a vertical cam track 24b and a second angled cam track 24c. The first angled cam track 24a is formed by traversing the cam profile from the vertex 11a to vertices 11b, 11c towards the proximal direction, a vertical cam track 24b is formed by traversing the cam profile from the vertex 11c to vertex 11d in the distal direction and second angled cam track 24c is formed by traversing the cam profile from the vertices 11d, 11e to vertex 11a again in the proximal direction. The cam profiles 10a, 10b may be in any polygon-shape for traversing of cam follower nuts 12a, 12b during the operation of trainer device 1.

Referring to FIG. 2, wherein each of cam profile 10a and 10b comprising two integral snap lock members 25 and 26, a first integral snap lock member 25 is formed by groove cut on the surface of cam track 24a and a second integral snap lock member 26 is formed by groove cut on the surface of cam track 24b. Further, the first snap lock member 25 comprising a protrusion head 25a towards distal end, which extends radially outward having the sloping surface 25b on the front projection surface and the second snap lock 26 member comprising a protrusion head 26a extend radially sideward having sloping surface 26b on the front projection surface. Both the first and second snap lock members 25, 26 are separated by a small protrusion raised upwardly. The integral snap lock members 25 and 26 provides the one-way traversing of cam follower nuts 12a and 12b in the cam profiles 10a, 10b during the operation of trainer device 1, while maintaining the cover member 5 in the first locking position 13 and second locking position 14.

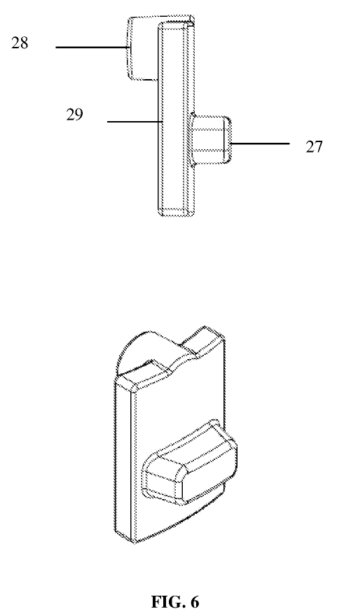

Referring to FIG. 6, the cam follower nuts 12a and 12b comprising a cam follower protrusion 27 on the distal end portion and a cam follower button 28 on the proximal end portion, whereas a cam follower body 29 is extending between 27 and 28. The protrusion 27 and the button 28 are extending opposite to each other from cam follower body 29.

Referring to FIGS. 1-6, the cam profiles 10a, 10b on the housing 2 along with the cam follower nuts 12a, 12b controls the movement of cover member 5 from the first locking position 13 to the second locking position 14. In the first locking position 13, the cover member 5 is in the retracted position and in the second locking position 14, the cover member 5 is in the extended position. The present disclosure provides single locking mechanism 9 that controls the cover member 5 in both the retracted and extended positions before and after use of trainer device 1.

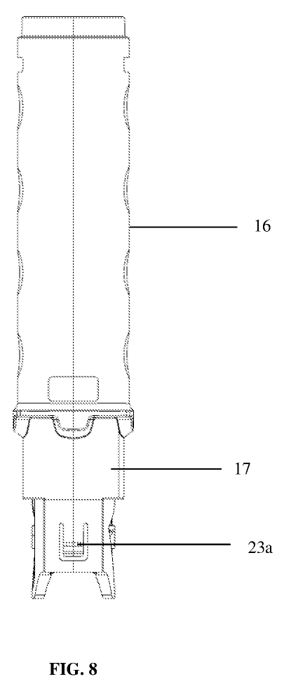

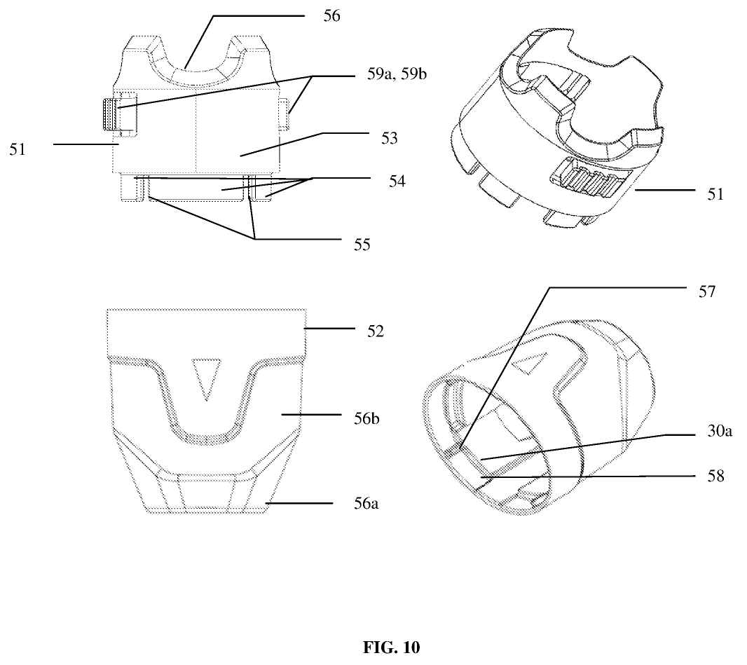

Referring to FIG. 2, and FIG. 8, locking projections 23a, 23b formed on the outer surface of second sleeve section 17 by groove cut diametrically opposite to the cam surfaces 22a, 22b. The locking projections 23a, 23b include a bevelled end surface extending radially outward which is shaped to lock with the reset members 59a, 59b (as shown in FIG. 10) of cover member 5 when the cover member 5 is in the extended position.

The cam follower button 28 of the cam follower nuts 12a and 12b is engaged with protrusion head 25a of cam track 24a in the first locking position 13. The cam follower button 28 of the cam follower nuts 12a and 12b abut with vertex 11d on the cam track 24b in the second locking position 14.

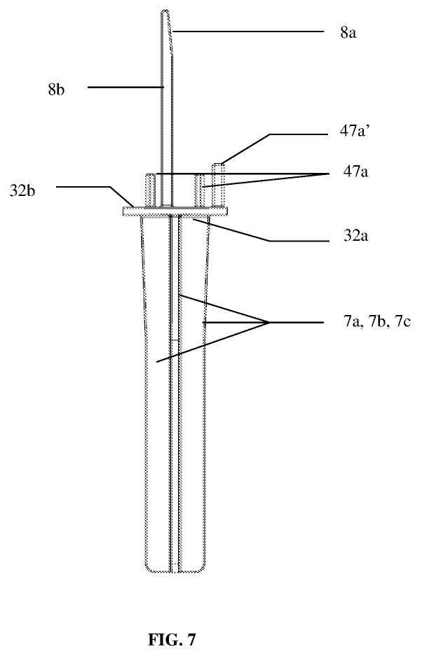

Referring to FIG. 1 and FIG. 7, a flex stem 7 comprising a body defining a longitudinal central axis, four longitudinal blades 7a, 7b, 7c, 7d extending radially outward from the body, and opposed proximal and distal end portions. Further, the flex stem 7 has a base part 32 having a front face 32a and a rear face 32b, whereas the four longitudinal blades 7a, 7b, 7c, 7d affixed to the front face 32a of the base part 32.

A flex arm 8 is extending from the rear face 32b of the flex stem base part 32 towards proximal end. A plurality of ribs 47a also extend from the rear face 32b of the flex stem base part 32. Furthermore, a rib 47a′ extends from the periphery of rear face 32b.

The flex arm 8 has a blade body having opposed first and second surfaces 8a, 8b that extend along a longitudinal axis of blade body with tapered end on the first surface 8a of the blade body.

Prior to the use of trainer device 1, the pin 50 of the safety cap 4 abuts with the tapered end of the flex arm 8 and obstructs the traversing of the flex arm 8 on the track 31 of the collet holder 3.

The flex stem 7 has abutment with the cover member 5 and is held against the spring 6 between the collet holder 3 and cover member 5, which restricts the movement of cover member 5 prior to the use of trainer device 1.

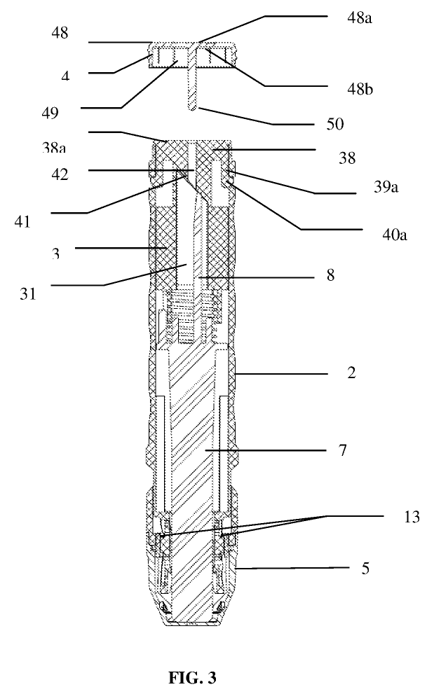

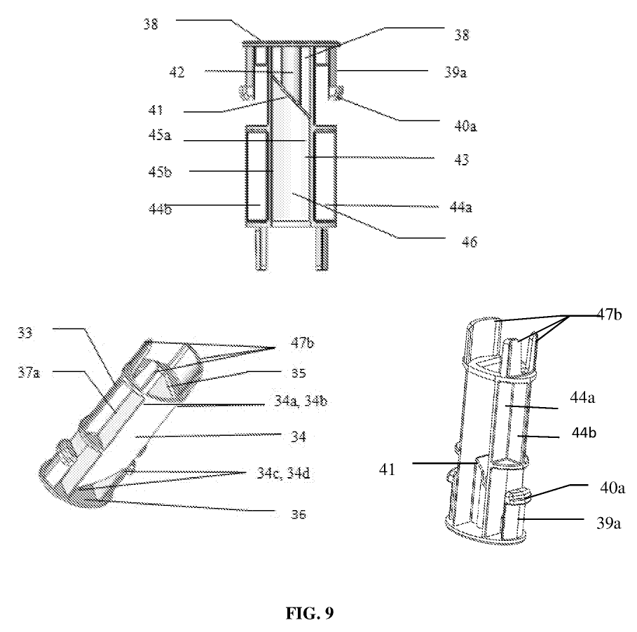

Referring to FIG. 3 and FIG. 9, the collet holder 3 is a single piece body defining longitudinal central axis having, opposed upper and bottom surfaces 33 and 34, opposed open distal end 35 and closed proximal end 36, a pair of laterally opposed sides 37a, 37b. Further, the collet holder 3 has an upper core member 38 with a top planar surface 38a perpendicular to longitudinal central axis.

The collet holder 3 further comprises a pair of opposed projecting clips 39a, 39b extending downwardly from the upper core member 38 with radially outward protrusion heads 40a and 40b at the distal end. The collet holder 3 may be cylindrical, oval or elliptical in shape.

The bottom surface 34 of collet holder 3 having four corners 34a, 34b, 34c and 34d, and a stop wall 41. The stop wall 41 extending upwardly from the two corners 34c, 34d of bottom surface 34 and being deflected outwardly at a region somewhat below the top-platen of collet holder 3 and disposed in common horizontal plane of upper core member 38. The upper core member 38 has an opening 42 extending in a common horizontal plane at the central axis. Further, the upper core member 38 of the collet holder 3 has an opening 42 to receive the safety cap 4.

The upper surface 33 of the collet holder 3 has a stop wall 41 towards closed proximal end 36 and a recess 43 extending longitudinally from open distal end 35 to the stop wall 41, and the stop wall 41 having front contact surface 41a. The recess 43 with the stop wall 41 defining the track 31 for the flex arm 8. Furthermore, the recess 43 formed in the upper surface 33 of the collet holder 3 having, a pair of opposed side surfaces 45a, 45b and central surface 46 extending parallel to the bottom surface 34 of collet holder 3 and ends in the stop wall 41. The flex arm 8 at the distal end is in always contact and rests in the front contact surface 41a of stop wall 41.

The trainer device 1 of an autoinjector, wherein the track 31 is formed by a recess 43 which extends longitudinally from open distal end 35 to the closed proximal end 36.

The laterally opposite sides 37a, 37b at the distal end 35 of the collet holder 3 are having two inclined surfaces 44a, 44b perpendicular to each other.

A pair of diametrically opposed extended ribs 47b extended from the open distal end 35 of collet holder 3, wherein one of extended rib 47b is having curved cross-sectional recess at the middle of the rib body.

Referring to FIG. 7 and FIG. 9, the rib 47a′ of flex stem 7 and diametrically opposed extended ribs 47b of the collet holder 3 define the spring platform 15, wherein the rib 47a′ fits in the curved cross-sectional recess of one of the extended rib 47b. The spring platform 15 provides constant holding of spring 6 during the operation of the trainer device 1.

A safety cap 4 is removably connected to the collet holder 3. Referring to FIG. 1 and FIG. 3, the safety cap 4 comprises a flat top wall 48 having a top planar surface 48a, a bottom surface 48b, and a side wall 49. The side wall 49 extends downwardly from the periphery of flat top wall 48 to the open free end.

The side wall has inner surface 49a and outer surface 49b. The inner surface 49a side wall 49 is sized to rest on the end surface of housing 18. Furthermore, the safety cap 4 has a pin 50 extending downwardly from the bottom surface 48b of top wall 48 and removably engaged within an opening 42 in the upper core member 38 of the collet holder 3.

To actuate the trainer device 1, the user has to remove the safety cap 4 which allows the traversing of the flex arm 8 on the track 31 of the collet holder 3 during the operation.

Referring to FIG. 10, the cover member 5 has two parts, an upper part 51 and a lower part 52, whereas the upper part 51 and the lower part 52 of cover member are connected via plurality of rib and groove arrangements.

The upper part 51 of cover member 5 comprising, opposed proximal and distal end portions, whereas both the ends having the opening. A side wall 53 extending from a proximal end to a distal end around the periphery of the upper part, a plurality of ribs 54 separated by continuous grooves 55 extending downwardly along an inner circumference of the side wall 53 at the distal end in symmetrical arrangement. Further, a pair of diametrically opposed U-shaped like cross-sectional grooves 56 are provided on the side wall 53 at the proximal end.

The lower part 52 of cover member 5 comprising: opposed open proximal end and closed distal ends. A bottom wall 56a having a periphery defined the closed distal end and a side wall 56b. The side wall 56b, first extending in an annular fashion from the open proximal end followed by tapering frustoconical fashion and nests to periphery of bottom wall 56a.

A plurality of ribs 57 separated by continuous grooves 58 extending upwardly along the inner circumference of side wall 56b at the proximal end to receive the corresponding ribs 54 and grooves 55 in the proper orientation of upper part 51 and lower part 52 of cover member 5. Diametrically opposed ledges 30a, 30b provided on the inner circumference of side wall 56b below the grooves 58. The ledges 30a, 30b are having two edges. During the operation of trainer device 1, the protrusion 27 of the cam follower nuts 12a and 12b moves rotationally from one edge to another edge of ledge 30a and 30b.

The inside diameter of annular side wall 56b at the proximal end of lower part 52 of cover member 5 is approximately equal to the outer diameter annular side wall 53 at the distal end of upper part 51 of cover member 5.

Referring to FIG. 10, the reset members 59a, 59b are provided on the cover member 5, more particularly on the upper part 51 of the cover ember 5. The reset members 59a and 59b is an elongated arm having upper and lower surfaces formed by groove cut on the surface of cover member 5, the end portion of reset members 59a, 59b are locked with locking projections 23a, 23b of the housing 2 by abutting of lower surface of the reset member with bevelled end surface of locking projection.

For using the device, first the safety cap 4 is removed and placed the cover member 5 on the suitable part of the body, for example the upper leg, and is pressed. The cover member 5 moves backwards towards proximal end which results in a relative backward movement of the flex stem 7. While the flex stem 7 is moved backwards, the flex arm 8 slidably traverses the front contact surface 41a of stop wall 41 from side surface 45a to 45b of recess 43. After removing the cover member 5 from the body, the cover member 5 is extended forward towards distal end by the action of the spring 6.

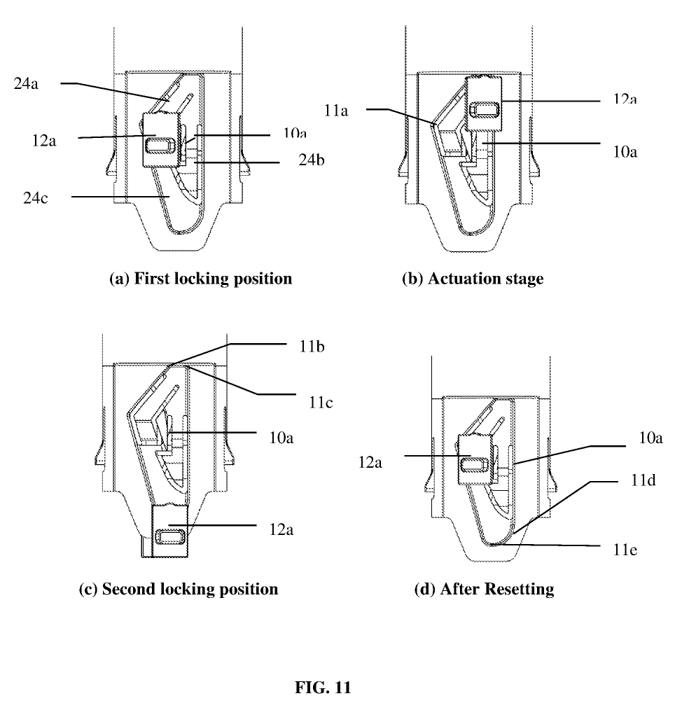

FIG. 11 shows the exemplary movement of cam follower nut 12a on the cam profile 10a during the various stages of the trainer device 1 for example the first locking position 13, the actuation stage, the second locking position 14 and the resetting stage. In the first locking position 13, the cam follower button 28 rests in the vertex 11a of cam track 24a. When the cover member 5 is moved backward, the cam follower button 28 traverses in the cam track 24a from vertex 11a to 11e via 11b; when the cover member 5 extends forward, the cam follower button 28 traverses in the cam track 24b from vertex 11e to vertex 11d and the cover member 5 is in the second locking position 14. Similarly, the cam follower nut 12b follows the same movement on the diametrically opposed cam profile 10b.

After using the trainer device 1, it may be ready for reuse with a resetting mechanism 60. As shown in FIG. 2 and FIG. 10, the resetting mechanism 60 comprises: locking projections 23a, 23b provided on the housing 2 and reset members 59a, 59b provided on the cover member 5. To reset the device, the user has to first place the safety cap 4 in the end surface 18 of housing 2 and presses the reset members 59a, 59b on cover member 5. Once the reset members 59a, 59b are pressed, the locking connection between the reset members 59a, 59b and locking projections 23a, 23b gets released and the bevelled end portion of locking projection easily passes the reset member; then user pushes the cover member 5 back towards the proximal end from the second locking position 14 to first locking position 13 and the trainer device 1 ready for reuse. The present disclosure provides resetting mechanism 60 and is separated from the locking mechanism 9 of cover member 5 and does not disturb the cover member 5 in both retracted and extended positions.

In the trainer device 1 resetting operation, the cover member 5 moves backwards to first locking position 13, as shown in FIG. 11, the cam follower button 28 traversing in cam track 24c from vertex 11d to 11a via 11e by passing of protrusion head 25a of first snap lock member 25.

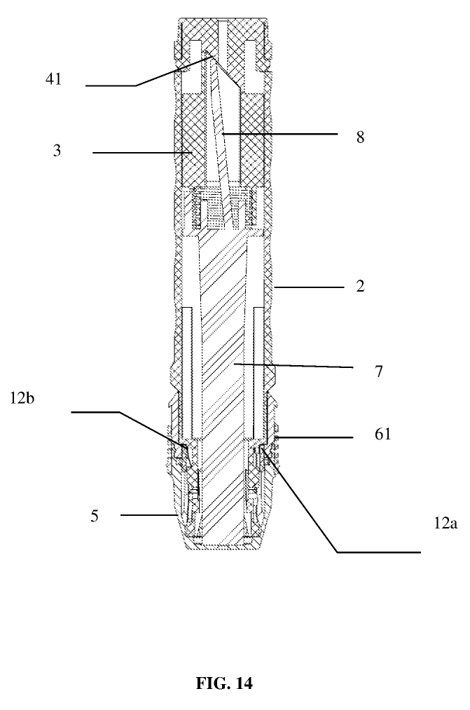

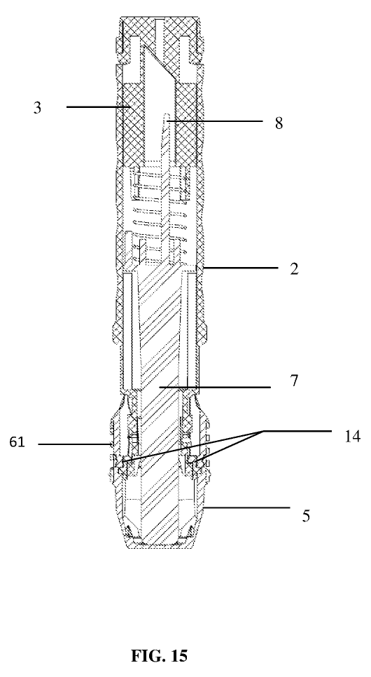

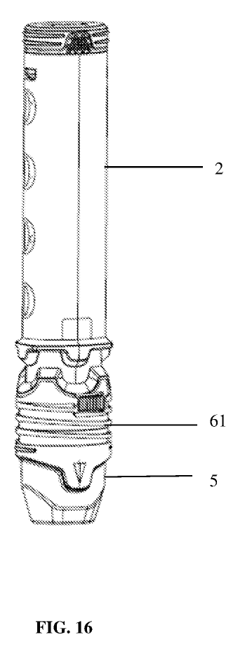

Another embodiment of the invention is described with reference to FIG. 2, FIG. 6, FIG. 7, FIG. 8, FIG. 9, FIG. 11 and FIGS. 12-23, a trainer device 1 for an autoinjector, comprising: a housing 2; a collet holder 3, a safety cap 4; a cover member 5; a flex stem 7; a flex arm 8; a cover member locking mechanism 9, wherein the housing 2 is partially received inside the cover member 5; wherein the cover member locking mechanism 9 comprising diametrically opposed cam profiles 10a, 10b and cam follower nuts 12a, 12b, wherein the cam follower nuts 12a,12b are positioned between the cover member and the housing 2 in both first locking position 13 and second locking position 14. Further the trainer device 1 comprising a spring 6 to bias the flex stem 7; the spring 6 is positioned between flex stem 7 and collet holder 3. The position of cover member 5 in first locking position 13 and second locking position 14 may be attained by cover member locking mechanism 9. The cover member 5 further comprises a sleeve, may be observed to be at least partially around the outer surface of the cover member 5.

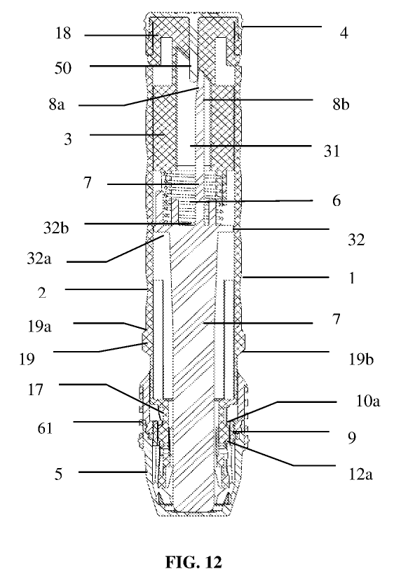

The housing 2 will now be described in greater detail in connection with FIG. 2, and FIG. 12. The housing 2 comprising a first sleeve section 16 and a second sleeve section 17, having opposed proximal and distal end portions, the second sleeve section 17 having reduced diameter than the first sleeve section 16 of the housing 2. The first sleeve section 16 at the proximal end has an end surface 18 to receive the safety cap 4 and the distal end has a peripheral ridge 19. The peripheral ridge 19 having front end 19a and rear end 19b portions, whereas the first sleeve section 16 is affixed into the front end portion 19a and the second sleeve section 17 extending from the rear end portion 19b. At the rear end 19b, a pair of diametrically opposed rectangular shaped like projection heads 20a, 20b, 20c, 20d extending downwardly on the surface of second sleeve section 17.

The second sleeve section 17 at distal end comprising diametrically opposed trapezium shaped like arms 21a, 21b which has cutting flank surfaces on the outer surface defining the cam surfaces 22a, 22b. The housing 2 may be cylindrical, oval or elliptical in shape.

The cam profiles 10a, 10b (as shown in FIG. 2 of aforementioned embodiment) are provided on the diametrically opposed cam surfaces 22a, 22b and each of the cam profile 10a and 10b is shape like a pentagon having five vertices 11a, 11b, 11c, 11d, and 11e. The cam profiles 10a, 10b comprising a first angled cam track 24a, a vertical cam track 24b and a second angled cam track 24c. The first angled cam track 24a is formed by traversing the cam profile from the vertex 11a to vertices 11b, 11c towards the proximal direction, a vertical cam track 24b is formed by traversing the cam profile from the vertex 11c to vertex 11d in the distal direction and second angled cam track 24c is formed by traversing the cam profile from the vertices 11d, 11e to vertex 11a again in the proximal direction. The cam profiles 10a, 10b may be in any polygon-shaped for traversing of cam follower nuts 12a, 12b during the operation of the trainer device 1.

Each of cam profile 10a and 10b comprising two integral snap lock members 25 and 26 (as shown in FIG. 2 of aforementioned embodiment), a first integral snap lock member 25 is formed by groove cut on the surface of cam track 24a and a second integral snap lock member 26 is formed by groove cut on the surface of cam track 24b. Further, the first snap lock member 25 comprising a protrusion head 25a towards distal end extend radially outward having the sloping surface 25b on the front projection surface and the second snap lock 26 member comprising a protrusion head 26a extend radially sideward having sloping surface 26b on the front projection surface. Both the first and second snap lock members 25, 26 are separated by a small protrusion raised upwardly. The integral snap lock members 25 and 26 provides the one-way traversing of cam follower nuts 12a and 12b in the cam profiles 10a, 10b during the operation of the trainer device 1, while maintain the cover member 5 in the first locking position 13 and second locking position 14.

The cam follower nuts 12a and 12b (as shown in FIG. 6 of aforementioned embodiment) comprising a cam follower protrusion 27 on the distal end portion and a cam follower button 28 on the proximal end portion, whereas a cam follower body 29 extending between 27 and 28. The protrusion 27 and the button 28 are extending opposite to each other from the cam follower body 29.

The cam profiles 10a, 10b on the housing 2 along with the cam follower nuts 12a, 12b controls the movement of the cover member 5 from the first locking position 13 to the second locking position 14. In the first locking position 13, the cover member 5 is in the retracted position and in the second locking position 14, the cover member 5 is in the extended position. The present disclosure provides single locking mechanism 9 that controls the cover member 5 in both the retracted and extended positions before and after use of trainer device 1.

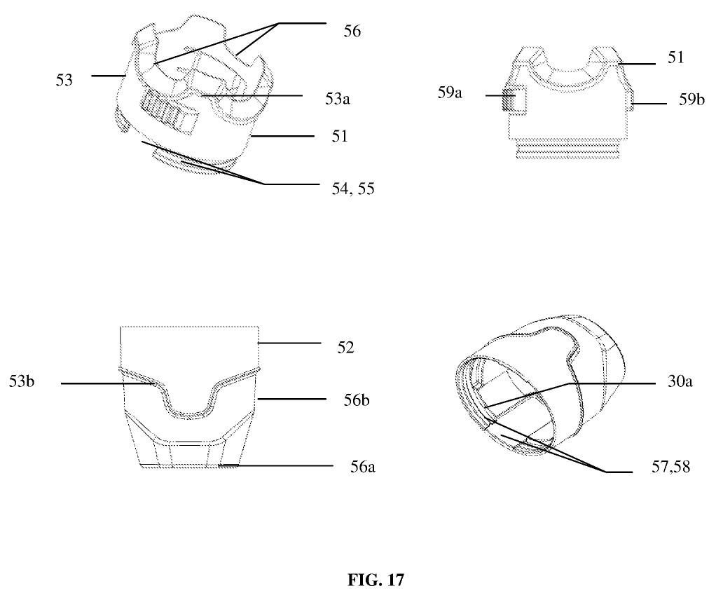

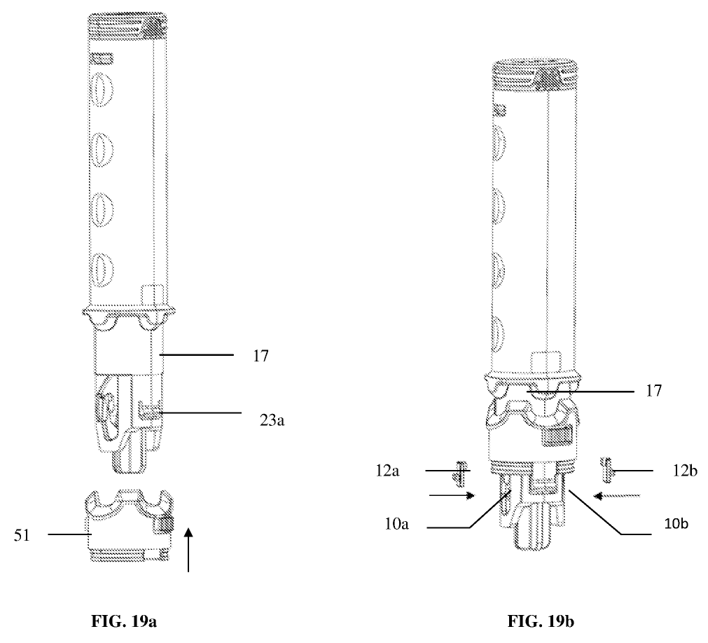

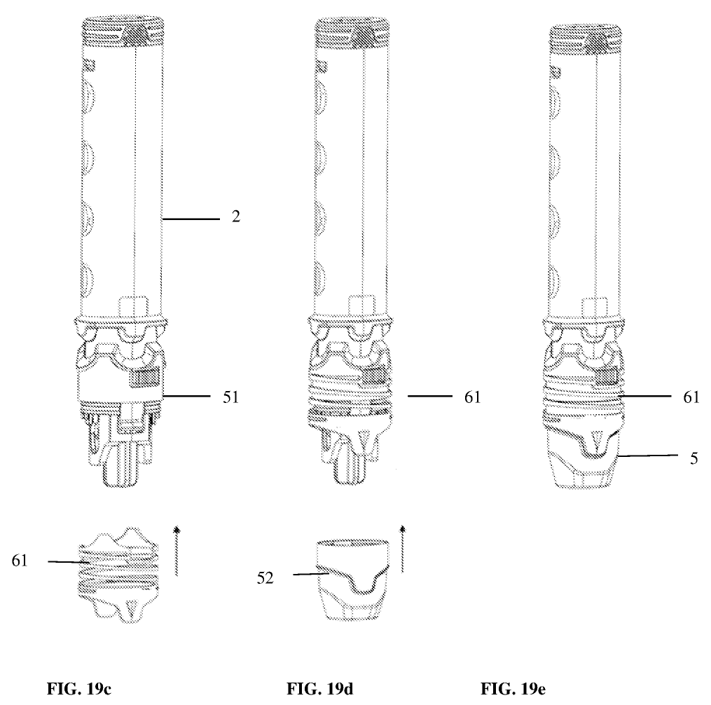

The locking projections 23a, 23b (as shown in FIG. 2, FIG. 8 of aforementioned embodiment and FIG. 19a) formed on the outer surface of second sleeve section 17 by groove cut diametrically opposite to the cam surfaces 22a, 22b. The locking projections 23a, 23b include a bevelled end surface extending radially outward which is shaped to lock with the reset members 59a, 59b (as shown in FIG. 17) of cover member 5 when the cover member 5 is in the extended position.

The cam follower button 28 of the cam follower nuts 12a and 12b is engaged with protrusion head 25a of cam track 24a in the first locking position 13. The cam follower button 28 of the cam follower nuts 12a and 12b abut with vertex 11d on the cam track 24b in the second locking position 14.

The flex stem 7 (as shown in FIG. 1 and FIG. 7 of aforementioned embodiment) comprising a body defining a longitudinal central axis, four longitudinal blades 7a, 7b, 7c, 7d extending radially outward from the body, and opposed proximal and distal end portions. Further, the flex stem 7 has a base part 32 having a front face 32a and a rear face 32b, whereas the four longitudinal blades 7a, 7b, 7c, 7d affixed to the front face 32a of the base part 32.

A flex arm 8 extends from the rear face 32b of the flex stem base part 32 towards the proximal end. A plurality of ribs 47a also extend from the rear face 32b of the flex stem base part 32. Furthermore, a rib 47a′ extends from the periphery of rear face 32b.

The flex arm 8 having a blade body having opposed first and second surfaces 8a, 8b that extends along a longitudinal axis of the blade body with the tapered end on the first surface 8a of the blade body.

Prior to the use of trainer device 1, the pin 50 of the safety cap 4 abuts with the tapered end of the flex arm 8 and obstructs the traversing of the flex arm 8 on the track 31 of the collet holder 3.

The flex stem 7 has abutment with the cover member 5 and is held against spring 6 between the collet holder 3 and the cover member 5, which restricts the movement of the cover member 5 prior to the use of trainer device 1.

The collet holder 3 (as shown in FIG. 3 and FIG. 9 of aforementioned embodiment) is a single piece body defining longitudinal central axis having, opposed upper and bottom surfaces 33 and 34, opposed open distal end 35 and closed proximal end 36, a pair of laterally opposed sides 37a, 37b, and an upper core member 38 with a top planar surface 38a perpendicular to longitudinal central axis.

The collet holder 3 further comprises a pair of opposed projecting clips 39a, 39b extending downwardly from the upper core member 38 with radially outward protrusion heads 40a and 40b at the distal end. The collet holder 3 may be cylindrical, oval or elliptical in shape.

The bottom surface 34 of collet holder 3 having four corners 34a, 34b, 34c and 34e, a stop wall 41 extending upwardly from the two corners 34c, 34d of bottom surface 34 and being deflected outwardly at a region somewhat below the top-platen of collet holder 3 and disposed in common horizontal plane of upper core member 38. The upper core member 38 having an opening 42 extends in a common horizontal plane at the central axis.

Further, the upper core member 38 of collet holder 3 has an opening 42 to receive the safety cap 4.

The upper surface 33 of the collet holder 3 having a recess 43 extending longitudinally from open distal end 35 to the closed proximal end 36 comprising a stop wall 41, and the stop wall 41 having front contact surface 41a. The recess 43 with the stop wall 41 defining the track 31 for the flex arm 8. Furthermore, the recess 43 formed in the upper surface 33 of the collet holder 3 having, a pair of opposed side surfaces 45a, 45b and central surface 46 extending parallel to the bottom surface 34 of collet holder 3 and ends in the stop wall 41. The flex arm 8 at the distal end is in always contact and rests in the front contact surface 41a of stop wall 41.

The trainer device 1 of an autoinjector wherein the track 31 may be formed by a recess 43 extends longitudinally from open distal end 35 to the closed proximal end 36.

At the distal end 35 of the collet holder 3, the laterally opposite sides 37a, 37b have two inclined surfaces 44a, 44b perpendicular to each other.

A pair of diametrically opposed extended ribs 47b extended from the open distal end 35 of the collet holder 3, wherein one of the extended rib 47b having curved cross-sectional recess at the middle of the rib body.

The rib 47a′ of the flex stem 7 and diametrically opposed extended ribs 47b of the collet holder 3 define the spring platform 15 (as shown in FIG. 7 and FIG. 9 of aforementioned embodiment), wherein the rib 47a′ fits in the curved cross-sectional recess of one of the extended rib 47b. The spring platform provides constant holding of the spring 6 during the operation of trainer device 1.

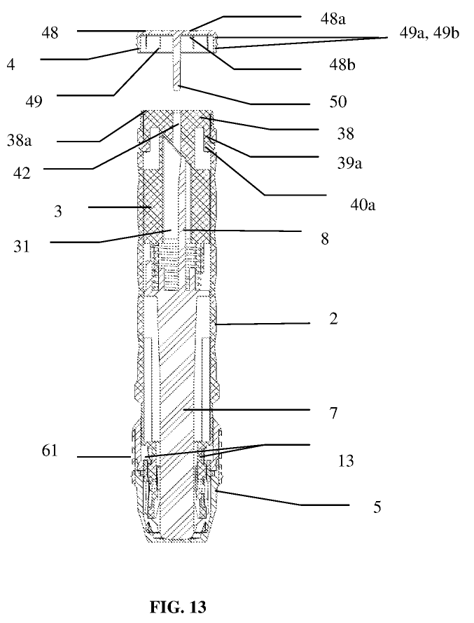

A safety cap 4 is removably connected to the collet holder 3 (as shown in FIG. 13), the safety cap 4 comprises a flat top wall 48 having top planar surface 48a, a bottom surface 48b, a side wall 49 extending downwardly from the periphery of flat top wall 48 to the open free end and the inner surface 49a and outer surface 49b. The inner surface 49a side wall 49 is sized to rest on the end surface 18 of housing 2. Furthermore, the safety cap 4 comprising a pin 50 extending downwardly from the bottom surface 48b of top wall 48 and removably engaged within an opening 42 in the upper core member 38 of the collet holder 3.

To actuate the trainer device 1, the user has to remove the safety cap 4 which allows the traversing of the flex arm 8 on the track 31 of the collet holder 3 during the operation.

As shown in FIG. 17, the cover member 5 has two parts, an upper part 51 and a lower part 52, whereas the upper part 51 and lower part 52 of cover member are connected via plurality of rib and groove arrangements.

The upper part 51 of cover member 5 comprising, opposed proximal and distal end portions, whereas both the ends having the opening. A side wall 53 extending from a proximal end to a distal end around the periphery of the upper part, a plurality of ribs 54 separated by continuous grooves 55 extending downwardly along an inner circumference of the side wall 53 at the distal end in symmetrical arrangement. Further, a pair of diametrically opposed U-shaped like cross-sectional grooves 56 are provided on the side wall 53 at the proximal end.

The annular side wall 53 provides an outer bearing surface and having stopping ridge 53a around the periphery of the upper part 51 of cover member 5 at the proximal end.

The lower part 52 of the cover member 5 comprising an opposed open proximal end and closed distal end. A bottom wall 56a has a periphery defining the closed distal end and a side wall 56b first extending in an annular fashion from the open proximal end followed by tapering frustoconical fashion and nests to periphery of bottom wall 56a. The side wall 56b provides an outer bearing surface and having stopping ridge 53b.

A plurality of ribs 57 separated by continuous grooves 58 extending upwardly along the inner circumference of side wall 56b at the proximal end to receive the corresponding ribs 54 and grooves 55 in the proper orientation of upper part 51 and lower part 52 of the cover member 5. Diametrically opposed ledges 30a, 30b provided on the inner circumference of side wall 56b below the grooves 58. The ledges 30a, 30b having two edges. During the operation of trainer device 1, the protrusion 27 of the cam follower nuts 12a and 12b moves rotationally from one edge to another edge of ledges 30a and 30b.

The inside diameter of annular side wall 56a at the proximal end of lower part 52 of the cover member 5 is approximately equal to the outer diameter annular side wall 53 at the distal end of upper part 51 of cover member 5.

In one preferred embodiment, the sleeve around the outer surface of the cover member 5 is a plastic sleeve 61 (as shown in FIGS. 12-16).

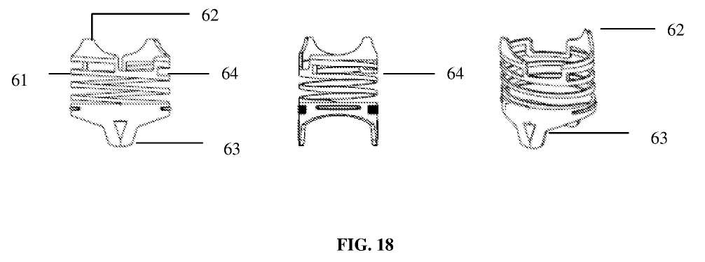

The plastic sleeve 61 will now be described in greater detail in connection with FIG. 18. The sleeve 61 may be a friction reducing sleeve provided over the cover member 5. The plastic sleeve 61 may comprise plastic materials but not limited to thermosetting epoxy, polyimide, polyether ketone, polyester resins combinations thereof, and/or the like. The plastic sleeve 61 may include both a first end 62 and a second end 63, wherein the first and second ends (62, 63) are separated by coil body 64 extending along a coiled axis. The coil body 64 comprising a fibre-reinforced polymer material being substantially wound in one direction only helically and concentrically at prescribed winding angle.

The first end 62 of plastic sleeve 61 may be positioned around the bearing surface of the annular side wall 53 of upper part 51 of the cover member 5 and the stopping ridge 53a prevents or inhibits the plastic sleeve 61 from extending beyond the stopping ridge 53a. The second end 63 of plastic sleeve 61 may be positioned around the bearing surface of side wall 56b of lower part 52 of the cover member 5 and the stopping ridge 53b prevents or inhibits the plastic sleeve 61 from extending beyond the stopping ridge 53b.

The plastic sleeve 61 may be at least partially coextensive with the cover member 5, wherein the plastic sleeve 61 has friction-reducing internal surface relative to the outer bearing surface of both the upper part 51, lower part 52 of the cover member 5 and is freely slidable between stopping ridges 53a and 53b.

As described herein, the trainer device 1 should be grasped proximal to the cover member 5. The plastic sleeve 61 has an external surface against which the user's hand can be positioned as the user accidentally grasps the upper part 51 of the cover member 5 during actuation of the trainer device 1. The plastic sleeve 61 may be in stationary position relative to user grasping, since the internal surface of the plastic sleeve 61 has low coefficient of friction that has slidable fit with the cover member 5 which allows the backward movement of the cover member 5 toward proximal end without any obstruction.

The coil body 64 acts as a helical spring and allows the compression of sleeve 61 when the proximal end 62 and the distal end 63 of the sleeve 61 are urged toward each other when the cover member 5 slidably moves towards proximal end. As the cover member 5 moves backward, the sleeve 61 gets compressed by the coil body 64 and maintains the sleeve 61 in constant contact with the cover member 5.

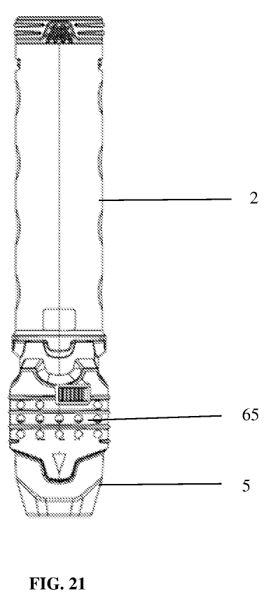

In another preferred embodiment, the sleeve around the outer surface of the cover member 5 is an elastomeric sleeve (as shown in FIG. 21).

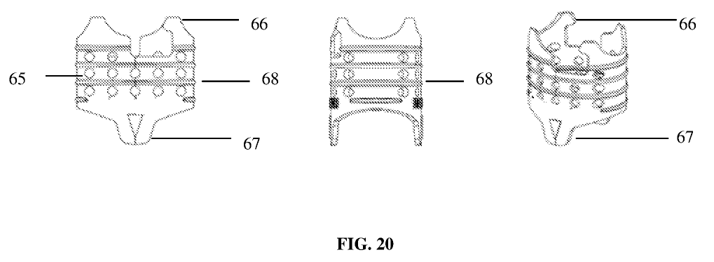

Referring to FIG. 20, the elastomeric sleeve 65 may be an elongated friction reducing sleeve. The elastomeric sleeve 65 may be a friction reducing sleeve provided over the cover member 5. The internal diameter and an internal surface of the elastomeric sleeve 65 that together provide a close but freely slidable fit with the cover member 5. Referring to FIG. 20, the elastomeric sleeve 65 may include both a first end 66 and a second end 67, wherein the first and second ends (66, 67) are separated by a tubular body 68 and the first and second ends may include openings.

The first end 66 of elastomeric sleeve 65 may be positioned around the bearing surface of the annular side wall 53 of upper part 51 of cover member 5 and the stopping ridge 53a prevents or inhibits the elastomeric sleeve 65 from extending beyond the stooping ridge 53a. The second end 67 of elastomeric sleeve 65 may be positioned around the bearing surface of side wall 56b of lower part of cover member 5 and the stopping ridge 53b prevents or inhibits the elastomeric sleeve from extending beyond the stopping ridge 53b.

The elastomeric sleeve 65 may be at least partially coextensive with the cover member 5, wherein the elastomeric sleeve 65 has friction-reducing internal surface relative to the outer bearing surface of both the upper part 51, lower part 52 of cover member 5 and is freely slidable between stopping ridges 53a and 53b.

The elastomeric sleeve 65 may be formed of any suitable material that maintains low coefficient of static friction on both internal and external surfaces and resilient function. The desired coefficients of friction are below 0.5, and are preferably below 0.1.

The elastomeric sleeve 65 has an external surface against which the user's hand can be positioned as the user accidentally grasps the upper part 51 of cover member 5 during actuation of the trainer device 1. The elastomeric sleeve 65 is in stationary position relative to user grasping, since the internal surface of the elastomeric sleeve 65 has slidable fit with the cover member 5 which allows the backward movement of the cover member 5 toward proximal end without any obstruction.

The tubular body 68 may be in elastomeric nature which acts as a spring and causes the compression of the sleeve 65 when the first end 66 and second end 67 of elastomeric sleeve 65 are urged towards each other when the cover member 5 slidably move towards proximal end of trainer device 1. As the cover member 5 moves backward, the sleeve 65 gets compressed by the tubular elastomeric body 68 and maintains the sleeve 65 in constant contact with the cover member 5.

After removing the cover member 5 from the body, the cover member 5 is extended forward towards distal end by the action of spring 6. As the cover member 5 extended forwards, the elastomeric sleeve 65 is slipping from the user's hand and traverse with the cover member 5.

During forward movement of the cover member 5, the elastomeric sleeve 65 remains stationary relative to the outer bearing surface of cover member 5.

The elastomeric sleeve 65 may comprise suitable material such as thermoset elastomers, for example natural rubbers, synthetic rubbers, thermoplastic elastomers and durable synthetic plastic material. As examples of synthetic rubbers, styrene-butadiene rubber, nitrile rubber, neoprene and ethylene-propylene rubbers, including copolymers and terpolymers such as ethylene-propylene terpolymer, can be mentioned. Examples of thermoplastic elastomers are styrene-butadiene-styrene, styrene-isoprene-styrene and styrene-ethylene-butylene block copolymers, as well as nylon types and olefinic, polyolefinic, polyester and polyurethane types.

The synthetic rubber also includes synthetic polyisoprene, polybutadiene, butadiene-styrene copolymers, butadiene-acrylonitrile copolymers, butadiene styrene acrylonitrile copolymers, polychloroprene and similar rubber products of 1,3-diene monomers having a markedly reduced coefficient of friction.

The surface of the elastomeric or rubber article is directly treated and the surface structure thereof is modified whereby the coefficient of friction of the rubber surface is greatly reduced without alternating the essential elastic properties of the rubber article.

Suitable low friction coatings include self-lubricating materials, such as synthetic fluoropolymer, Nylon, or any other suitable low friction material or coating.

Examples of synthetic plastics materials are polyvinyl chloride and polyethylene. A plasticiser can be added to a plastic material makes sleeve flexible, and resilient in function. Examples of plasticisers are low molecular weight phthalates, high molecular weight phthalates and speciality plasticisers such as adipates, citrates, benzoates, trimelilates and polyoxyethylene additives such as acetal copolymers, polyacetal and polyformaldehyde.

The plastic sleeve 61 or an elastomeric sleeve 65 has a substantially circular or oval cross section.

The assembling procedures of the cover member 5 and the plastic sleeve 61 to the housing 2 of the trainer device 1 will now be described in greater detail in connection with FIGS. 19a-19e. The upper part 51 of the cover member 5 is first positioned over the second sleeve section of housing 17 and securely placing the cam follower nuts 12a and 12b on the corresponding cam profiles 10a and 10b such that the cam follower buttons 28 are engaged with the first snap lock member 24. The first end of the plastic sleeve 61 or the elastomeric sleeve 65 is affixed into the bearing surface of the upper part 51 of the cover member 5 and the stopping ridge 53a provides that the plastic sleeve 61 or the elastomeric sleeve 65 does not extend beyond the outer periphery of the upper part 51 of the cover member 5. After fixing the sleeve 61 or 65, the lower part 52 is positioned into the upper part 51 of the cover member 5 and gets connected via plurality of rib and groove arrangements, such that the second end of the sleeve 61 or 65 is received over the outer surface of lower part 52 of the cover member 5. The cam follower protrusions 27 are received in the ledges 30a, 30b of the lower part 52 of the cover member 5.

The assembling procedures of the cover member 5 and the elastomeric sleeve 65 to the housing 2 of the trainer device 1 will be in similar manner as described in FIGS. 19a-19e.

As depicted in FIG. 11 of aforementioned embodiment, the cam follower nut 12a moves on the cam profile 10a during the various stages of the trainer device 1 for example the first locking position 13, the actuation stage, the second locking position 14 and the resetting stage. In the first locking position 13, the cam follower button 28 rests in the vertex 11a of cam track 24a. When the cover member 5 is moved backward, the cam follower button 28 traverses in the cam track 24a from vertex 11a to 11e via 11b; when the cover member 5 extends forward, the cam follower button 28 traverses in the cam track 24b from vertex 11e to vertex 11d and the cover member 5 is in the second locking position 14. Similarly, the cam follower nut 12b follows the same movement on the diametrically opposed cam profile 10b.

As described herein, for using the trainer device 1, the safety cap 4 has to be removed first and the cover member 5 needs to be placed on the suitable part of the body, for example the upper leg, and is pressed. The lower part 52 and upper part 51 of the cover member 5 moves backwards towards the proximal end which results in a relative backward movement of the flex stem 7. While the flex stem 7 is moved backwards, the flex arm 8 slidably traverses the front contact surface 41a of stop wall 41 from side surface 45a to 45b of recess 43. After removing the cover member 5 from the body, the cover member 5 extends forward towards the distal end by the action of spring 6.

After using the trainer device 1, it may be ready for reuse with a resetting mechanism 60. The resetting mechanism 60 comprising: locking projections 23a, 23b provided on the housing 2 (as shown in FIG. 19a) and reset members 59a, 59b provided on the outer sleeve 73 of top cover member 70 (as shown in FIG. 17).

As shown in FIG. 17, the reset members 59a and 59b is an elongated arm having upper and lower surfaces formed by groove cut on the surface of upper part 51 of the cover member 5, the end portion of the reset members 59a, 59b are locked with locking projections 23a, 23b by abutting of lower surface of reset members 59a, 59b with bevelled end surface of locking projection.

To reset the device, the user has to first place the safety cap 4 in the end surface 18 of housing 2 and presses the reset members 59a, 59b on the cover member 5. Once the reset members 59a, 59b are pressed, the locking connection between the reset members 59a, 59b and the locking projections 23a, 23b is released and the bevelled end portion of locking projection easily passes the reset member; then user pushes the cover member 5 back towards the proximal end from the second locking position 14 to first locking position 13 and the trainer device 1 ready for reuse. The present disclosure provides the resetting mechanism 60 which is separate from the locking mechanism 9 of the cover member 5 and does not disturb the cover member 5 in both retracted and extended positions.

In the trainer device 1 resetting operation, the cover member 5 moves backwards to first locking position 13, as shown in FIG. 11 of aforementioned embodiment, the cam follower button 28 is traversing in cam track 24c from vertex 11d to 11a via 11e by passing of protrusion head 25a of first snap lock member 25.

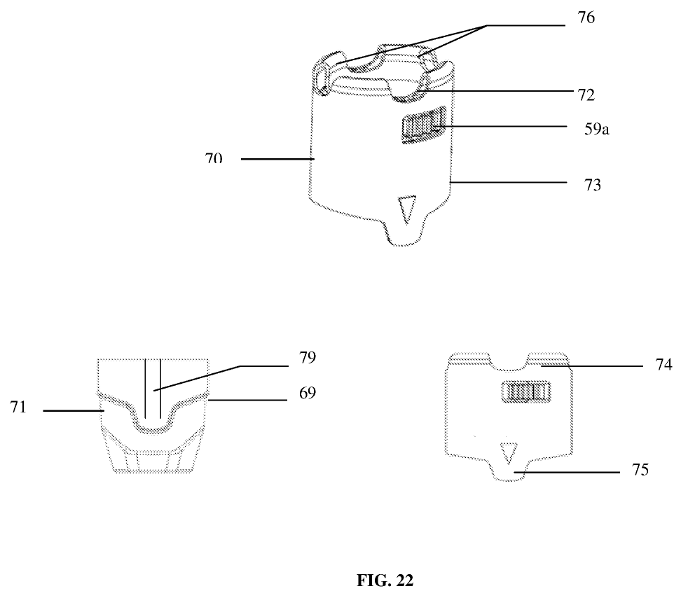

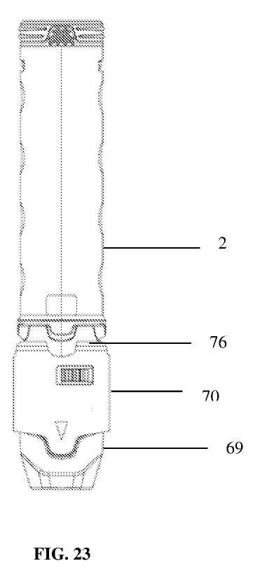

FIG. 22 and FIG. 23 depict an alternative embodiment of the cover member 5. The cover member 5 has a base cover member 69 and a top cover member 70. The base cover member 69 having opposed proximal and distal end portions, whereas both the ends having the opening and a side wall 71 extending from the proximal end to distal end. The top cover member 70 comprising an inner sleeve 72 and an outer sleeve 73, arranged coaxially and concentrically. The top cover member 70 may include both a first 74 and a second end 75. The concentrically arranged inner sleeve 72 and outer sleeve 73 are jointed together with an extending elastomeric part 76 at the first end 74. The inner sleeve 72 of top cover member 70 comprising plurality of ribs 77a separated by continuous grooves 77b extending downwardly along an inner circumference at the distal end in symmetrical arrangement.

A plurality of ribs and continuous grooves 78a, 78b (not shown) extending upwardly along the inner circumference of base cover member 69 at the proximal end to receive the corresponding ribs and grooves 77a, 77b (not shown) in the proper orientation of the base cover member 69 and the top cover member 70.

The inside diameter of annular side wall 71 of base cover member 69 is substantially equal to the outer diameter of inner sleeve 72 of top cover member 70 in order to facilitate snap fit.

The cover member 5 further comprising a pair of diametrically opposed guiding channels 79 running on the outer surface of inner sleeve 72 of the top cover member 70 and continuous on the outer surface of the base cover member 69. The guiding channels 79 extending from below the U-shaped like cross-sectional grooves 78 on the inner sleeve 72 of the top cover member 70 at the first end 74 to second end 75, since the inside diameter of annular side wall 71 of base cover member 69 is substantially equal to the outer diameter of inner sleeve 72 of top cover member 70 allowing snap fit between them, the guiding channels 79 continues on the outer surface of the base cover member 69.

The outer sleeve 73 of the top cover member 70 has a pair of diametrically opposed ribs 80 (not shown) on the inner surface corresponding to the guiding channels 79 on the outer surface of inner sleeve 72 of top cover member 70 and base cover member 69. The ribs 80 and the guiding channels 79 arrangements facilitate the movement of outer sleeve 73 of top cover member 70 during the actuation of trainer device 1.

The outer sleeve 73 of the top cover member 70 has an external surface against which the user's hand can be positioned as the user accidentally grasps the cover member 5 during actuation of the trainer device 1. The outer sleeve 73 of the top cover member 70 is in stationary position relative to user grasping, since the outer sleeve 73 has joint with the inner sleeve 72 by the elastomeric part 76, the ribs 80 and guiding channels 79 permit the movement of the inner sleeve 72 backwardly towards the proximal end by stretching of the elastomeric part 76 along with the base cover member 69.

After removing the cover member 5 from the body, the cover member 5 is extended forward towards the distal end by the action of the spring 6. As the inner sleeve 72 of the top cover member 70 along with the base cover member 69 extend forwards, the outer sleeve 73 slips from the user's hand and traverses with both the inner sleeve 72 and the base cover member 69.

Various modifications and variations to the above-described trainer device 1 can be made without departing from the scope of the present disclosure.

We claim:

1. A trainer device for an autoinjector comprising: (i) a housing; (ii) a collet holder that includes a track; (iii) a safety cap removably connected to the collet holder; (iv) a cover member; (v) a flex stem having flex arm; and (vi) a cover member locking mechanism having a first locking position and a second locking position, wherein the housing is partially received inside the cover member, and the flex arm of the flex stem traverses the track during actuation of the trainer device upon removal of the safety cap.

2. The trainer device of claim 1, wherein the cover member locking mechanism comprises cam profiles and cam follower nuts.

3. The trainer device of claim 2, wherein the cam profiles are provided on the housing.

4. The trainer device of claim 2, wherein the cam follower nuts are positioned between the cover member and the housing.

5. The trainer device of claim 2, wherein the cam profile has five vertices and comprises a first angled cam track, a vertical cam track and a second angled cam track: wherein (i) the first angled cam track is formed by traversing of the cam profile from the first vertex to second and third vertices towards the proximal direction; (ii) the vertical cam track is formed by traversing of the cam profile from the third vertex to fourth vertex in the distal direction; and (iii) the second angled cam track is formed by traversing the cam profile from the fourth and fifth vertices to first vertex again in the proximal direction.

6. The trainer device of claim 2, wherein the cam profile comprises two integral snap lock members which include (i) first integral snap lock member formed by groove cut on the surface of first angled cam track, and (ii) second integral snap lock member formed by groove cut on the surface of vertical cam track.

7. The trainer device of claim 2, wherein the cam follower nuts comprise a cam follower protrusion on the distal end portion; a cam follower button on the proximal end portion; and a cam follower body extending between protrusion and button, wherein the protrusion and button are extending opposite to each other.

8. The trainer device of claim 7, wherein the cam follower button of the cam follower nuts are engaged with the cam track in the first locking position.

9. The trainer device of claim 7, wherein the cam follower button of the cam follower nuts are engaged with the fourth vertex on the cam track in the second locking position.

10. The trainer device of claim 1, wherein the collet holder comprises (i) a single piece body defining longitudinal central axis having opposed upper and bottom surfaces, (ii) opposed open distal end and a closed proximal end, (iii) a pair of laterally opposed sides, and (iv) an upper core member with a top planar surface perpendicular to longitudinal central axis.

11. The trainer device of autoinjector of claim 10, wherein the upper surface of the collet holder has a stop wall and a recess, wherein the recess extends longitudinally from the open distal end to the stop wall and defining the track for the flex arm.

12. A trainer device for an autoinjector comprising (i) a housing; (ii) a collet holder; (iii) a safety cap removably connected to the collet holder; (iv) a flex stem having flex arm; (v) a cover member, and (vi) a cover member locking mechanism having a first locking position and a second locking position, wherein in the first locking position, the cover member is in the retracted position and in the second locking position, the cover member is in the extended position; said cover member locking mechanism comprising cam profiles and cam follower nuts, wherein the cam profiles are provided on the cam surfaces of housing and cam follower nuts are positioned between the cover member and the housing in both first locking position and second locking position; and (vii) a resetting mechanism comprising locking projections provided on the housing and reset members provided on the cover member, wherein the resetting of cover member results in proximal movement of cover member to the retracted position; said proximal movement is allowed by pressing the reset members that release the locking connection between the reset members and locking projections.

13. A trainer device for an autoinjector comprising: a housing and a cover member, wherein the cover member further comprises a sleeve which is at least partially surrounds the outer surface of the cover member to prevent or inhibit the obstruction of the movement of the cover member when the user accidentally holds the cover member during the actuation of trainer device.

14. The trainer device of claim 13, wherein the sleeve is a plastic sleeve or an elastomeric sleeve.

15. The trainer device of claim 14, wherein the plastic sleeve includes a first end and a second end, wherein the first end and second end are separated by a coil body extending along a coiled axis.
